(19) United States
(12) Patent Application Publication
(10) Pub. No.: US 2024/0188856 A1
(43) Pub. Date: Jun. 13, 2024

(54) ELECTRONIC DEVICE AND METHOD OF DETERMINING ACCURACY OF ANALYTE CONCENTRATION

(71) Applicant: SAMSUNG ELECTRONICS CO., LTD., Suwon-si (KR)

(72) Inventor: Juneyoung LEE, Suwon-si (KR)

(73) Assignee: SAMSUNG ELECTRONICS CO., LTD., Suwon-si (KR)

(21) Appl. No.: 18/142,271

(22) Filed: May 2, 2023

(30) Foreign Application Priority Data
Dec. 9, 2022 (KR) 10-2022-0171868

(51) Int. Cl. A61B 5/145; A61B 5/00; A61B 5/1455; A61B 5/1495

(52) U.S. Cl. CPC A61B 5/14546; A61B 5/0075; A61B 5/1455; A61B 5/1495; A61B 5/7267

(57) ABSTRACT

An electronic device determines accuracy of an analyte concentration non-invasively by calculating an analyte concentration of an analyte from an in-vivo spectrum obtained non-invasively using a calibration model, generates a test calibration model based on feature vectors of the in-vivo spectrum obtained non-invasively from which a spectrum specific to the analyte have been subtracted, calculates a test concentration for the analyte from the in-vivo spectrum using the test calibration model, and analyzes the test concentration for the analyte using the test calibration model to determine the accuracy of the analyte concentration using the calibration model.

### CROSS-REFERENCE TO RELATED APPLICATION

This application is based on and claims priority under 35 U.S.C. § 119 to Korean Patent Application No. 10-2022-0171868, filed on Dec. 9, 2022, in the Korean Intellectual Property Office, the disclosure of which is incorporated by reference herein in its entirety.

### BACKGROUND

### 1. Field

The present disclosure relates to an electronic device and method for determining the accuracy of an analyte concentration.

### 2. Description of Related Art

An invasive method or a non-invasive method may be used to ascertain the concentration of an analyte of a test subject. The non-invasive method has an advantage in that it does not require invasive behavior. Spectroscopy such as absorption spectroscopy and Raman spectroscopy may be used for non-invasive methods. An in-vivo spectrum is obtained from measurements of absorption of spectrum by a test subject or measurements of scattering electromagnetic radiation by the test subject. The in-vivo spectrum includes information on biomolecules included in the test subject. The concentration of the analyte of the test subject can be calculated by calibrating the in-vivo spectrum. A true value of the analyte concentration obtained by the invasive method is used to analyze the accuracy of the analyte concentration calculated in the non-invasive manner. This has a disadvantage of requiring an invasive action.

### SUMMARY

Provided are an electronic device and method for determining the accuracy of analyte concentration according to various embodiments. The technical problem to be achieved by the present disclosure is not limited to the technical challenges as described above, and other technical challenges may be inferred from the following embodiments.

Additional aspects will be set forth in part in the description which follows and, in part, will be apparent from the description, or may be learned by practice of the presented embodiments of the disclosure.

According to an aspect of an embodiment, an electronic device for determining accuracy of an analyte concentration non-invasively includes a memory configured to store one or more instructions, and one or more processors, wherein the one or more processors execute the one or more instructions to calculate an analyte concentration from an in-vivo spectrum obtained non-invasively by using a calibration model, subtract a spectrum specific to the analyte from the in-vivo spectrum, generate a test calibration model based on feature vectors of the subtracted in-vivo spectrum obtained non-invasively, calculate a test concentration for the analyte from the in-vivo spectrum by using the test calibration model, and determine the accuracy of the analyte concentration calculated using the calibration model by analyzing the test concentration for the analyte using the test calibration model.

The one or more processors are configured to obtain a spectrum specific to the analyte by scaling a net spectrum of the analyte to the analyte concentration.

The one or more processors are configured to obtain a spectrum specific to the analyte by scaling a net spectrum of the analyte to the analyte concentration and an optical path length.

The one or more processors are configured to generate the test calibration model by calculating a net analyte signal (NAS) from feature vectors of the subtracted in-vivo spectrum.

The one or more processors are configured to obtain the feature vectors by performing feature extraction on the subtracted in-vivo spectrum.

The feature vectors of the subtracted in-vivo spectrum include main components obtained by performing principal component analysis (PCA) on the subtracted in-vivo spectrum.

The one or more processors are configured to determine the accuracy of the analyte concentration by comparing the test concentration for the analyte with a reference value.

The one or more processors are configured to determine the accuracy of the analyte concentration by comparing, with a reference value, a standard deviation of the test concentration for the analyte.

The one or more processors are configured to determine the accuracy of the analyte concentration by comparing, with a reference value, a standard deviation of the test concentration calculated for a predetermined interval.

The one or more processors are configured to determine that the accuracy of the analyte concentration is high when a standard deviation of the test concentration calculated for a 10-minute interval is less than 50 mg/dl.

The one or more processors are configured to determine the accuracy of the analyte concentration by comparing the test concentration for the analyte with the analyte concentration.

The calibration model is at least one of a PLS calibration model, a NAS calibration model, and a calibration model based on deep learning.

The test calibration model is a NAS calibration model.

The analyte is at least one of glucose, urea, lactic acid, triglyceride, protein, cholesterol, and ethanol.

The in-vivo spectrum is an in-vivo spectrum of a test subject generated based on absorption spectroscopy.

The in-vivo spectrum is an in-vivo spectrum of a test subject for near-infrared rays.

The one or more processors are configured to determine, in a non-invasive manner, the accuracy of the analyte concentration calculated using the calibration model.

According to another aspect of an embodiment, a method of determining accuracy of an analyte concentration non-invasively includes calculating the analyte concentration from an in-vivo spectrum obtained non-invasively by using a calibration model, subtracting a spectrum specific to an analyte from the in-vivo spectrum, generating a test calibration model based on feature vectors of the subtracted in-vivo spectrum obtained non-invasively, calculating a test concentration for the analyte from the in-vivo spectrum by using the test calibration model, and analyzing the test concentration for the analyte using the test calibration model to determine accuracy of the analyte concentration non-invasively.

The determining of the accuracy of the analyte concentration includes comparing the test concentration of the analyte with a reference value to determine the accuracy of the analyte concentration.

According to an aspect of an embodiment, provided is a computer-readable recording medium in which a program for executing the method in a computer is recorded.

### BRIEF DESCRIPTION OF THE DRAWINGS

The above and other aspects and features of example embodiments of the disclosure will be more apparent from the following description taken in conjunction with the accompanying drawings, in which:

FIG. 1 illustrates an electronic device for determining the accuracy of an analyte concentration according to an embodiment;

FIG. 2 illustrates a calibration model according to an embodiment;

FIG. 3 is a diagram illustrating a net analyte signal (NAS) calibration model according to an embodiment;

FIG. 4 is a diagram for explaining correction of the concentration of an analyte in a prediction section according to an embodiment;

FIG. 5 is a graphical diagram illustrating a method of subtracting a spectrum specific to an analyte from an in-vivo spectrum according to an embodiment;

FIG. 6 is a graphical diagram illustrating a feature vector according to an embodiment;

FIG. 7 is a flowchart illustrating a method for determining the accuracy of an analyte concentration according to an embodiment; and

FIGS. 8A and 8B illustrate graphs of concentrations and test concentrations of glucose calculated by experiments according to an embodiment.

FIGS. 9A and 9B illustrate graphs of concentrations and test concentrations of glucose calculated by additional experiments according to an embodiment.

### DETAILED DESCRIPTION

Reference will now be made in detail to example embodiments, examples of which are illustrated in the accompanying drawings, wherein like reference numerals refer to like elements throughout. In this regard, embodiments consistent with the disclosure may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The terms used in these embodiments are selected as widely used general terms as possible while considering functions in example embodiments, but these may vary depending on the intention or precedent of a technician skilled in the art, the emergence of a new technology, and the like. In addition, in certain cases, there are arbitrarily selected terms, and in this case, the meaning will be described in detail in the description of an example embodiment. Therefore, the terms used in such embodiments should be defined based on the specified meaning of the terms and the overall context of such embodiments.

Singular expressions include plural expressions unless the context clearly indicates otherwise. In addition, when a part “includes” a component, this means that it may contain other components, rather than excluding other components, unless otherwise stated. The wording “substantially the same” may be widely interpreted as encompassing the same and similar unless the context clearly indicates otherwise.

Hereinafter, embodiments will be described in detail with reference to the accompanying drawings so that a person of ordinary skill in the art may easily implement the embodiments. However, the inventive concept may be implemented in various different forms and is not limited to the embodiments described herein.

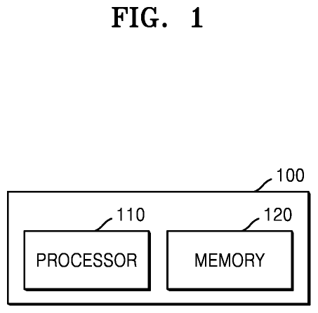

FIG. 1 illustrates an electronic device 100 for determining the accuracy of an analyte concentration according to an embodiment.

The electronic device 100 may be any electronic device including components for determining the accuracy of the analyte concentration. The electronic device 100 may be a computer, a personal computer (PC), a laptop, a wearable device, a blood glucose measurement device, or an experimental device, but is not limited thereto.

The electronic device 100 may include a processor 110 that performs instructions for determining the accuracy of the analyte concentration, and a memory 120 that stores the instructions. The processor 110 may be, but is not limited to, a central processing unit (CPU), an application processor (AP), a digital signal processor (DSP), a graphics processing unit (GPU), a vision processing unit (VPU), or a neural processing unit (NPU). The memory 120 may be an on-chip memory, a cache memory, random access memory (RAM), a read only memory (ROM), a solid state drive (SSD), a hard disk drive (HDD), or an optical disc drive (ODD), but is not limited thereto.

The electronic device 100 may further include components for calculating the analyte concentration. The electronic device 100 may include components for calculating the analyte concentration in a non-invasive method or an invasive method. The electronic device 100 may include components for calculating the analyte concentration in a non-invasive manner based on spectroscopy. Absorption spectroscopy or Raman spectroscopy may be used, but is not limited thereto. The electronic device 100 may include a light source and an optical detector. Any light source/optical detector capable of emitting/detecting the required electro-magnetic radiation may be used. For example, the required radiation may be Near Infrared (NIR), Short-Wavelength Infrared (SWIR), Mid-Wavelength Infrared (MWIR), or Long-Wavelength Infrared (LWIR), but is not limited thereto.

Hereinafter, embodiments of a method of determining accuracy of analyte concentration consistent with FIG. 1 will be described.

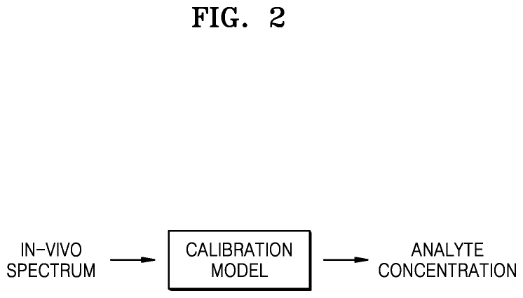

FIG. 2 illustrates a calibration model according to an embodiment.

A test subject may be a living body. An analyte may be any material constituting the test subject. The analyte may be a biomolecule included in a test subject. For example, the analyte may be at least one of glucose, urea, lactic acid, triglyceride, protein, cholesterol, or ethanol, but is not limited thereto.

An in-vivo spectrum may be a spectrum of electro-magnetic waves absorbed or scattered by the test subject. Radiation emitted from a light source to the test subject may be absorbed or scattered by biomolecules. Absorbed or scattered radiation may be detected by an optical detector. An in-vivo spectrum may be obtained from the detected radiation. Radiation of various wavelengths may be used to obtain an in-vivo spectrum by absorption. For example, NIR, SWIR, MWIR, or LWIR may be used, but the embodiments are not limited thereto. Elastic scattering or inelastic scattering may be used to obtain an in-vivo spectrum by scattering, but is not limited thereto.

A calibration model according to an embodiment is a model designed to calculate an analyte concentration from an in-vivo spectrum. The calibration model may be based on an algorithm that extracts a relatively low-dimensional space from a high-dimensional space of an in-vivo spectrum. For example, the calibration model may be, but is not limited to, a Partial Least Squares (PLS) calibration model or a Net Analyte Signal (NAS) calibration model. Furthermore, the calibration model may be based on a deep learning algorithm designed to infer analyte concentrations from in-vivo spectra. Neural networks of various types and structures may be used for the calibration model based on deep learning. For example, deep neural networks (DNNs), convolutional neural networks (CNNs), or recurrent neural networks (RNNs) may be used therefor, but are not limited thereto.

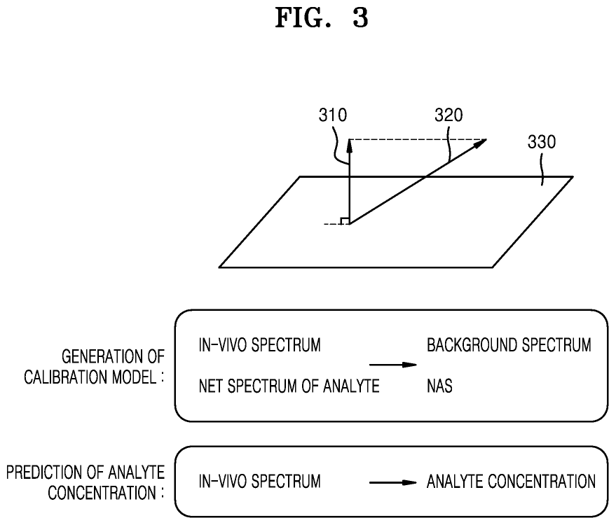

FIG. 3 is a diagram illustrating a net analyte signal (NAS) calibration model according to an embodiment.

The test subject may be composed of a target analyte and background components. In the NAS calibration model according to an embodiment, the background spectrum 330 refers to a spectrum indicating spectral variation according to the concentration of the background component in the in-vivo spectrum. A net spectrum 320 of the analyte means the spectrum of the analyte. A NAS 310 refers to a spectrum perpendicular to a background spectrum in the net spectrum of the analyte.

A NAS calibration model may be generated by calculating the background spectrum and the NAS from the in-vivo spectrum and the net spectrum of the analyte. In the generation of NAS calibration models, it is important how well the background spectrum and the NAS are calculated. To this end, it is preferable that a section in which the concentration of the analyte is constant is used as a learning section for generating a NAS calibration model. In a learning section where the concentration of the analyte is constant, spectral variation due to background components can be easily monitored. As an example, a background spectrum may be calculated by concatenating a set of in-vivo spectra in a learning interval in which the concentration of the analyte is constant. As an example, a NAS may be calculated by calculating a component perpendicular to the background spectrum from the net spectrum of the analyte.

The generated NAS calibration model may be used to calculate the concentration of the analyte with respect to the prediction interval. When an in-vivo spectrum is obtained for the prediction interval and input to the NAS calibration model, the analyte concentration for the prediction interval may be obtained from the NAS calibration model.

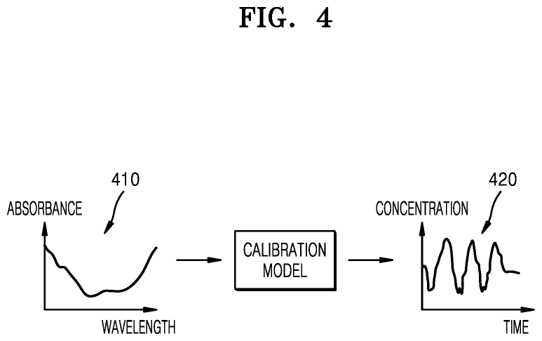

FIG. 4 is a diagram for explaining the correction of the concentration of an analyte in a prediction section according to an embodiment.

An arbitrary prediction interval for measuring the analyte concentration may be set, and an in-vivo spectrum 410 for the prediction interval may be obtained. An analyte concentration 420 for a prediction interval may be calculated from the in-vivo spectrum 410 by the calibration model.

In the left side of FIG. 4, the in-vivo spectrum 410 obtained for a prediction interval is shown. The in-vivo spectrum 410 may be obtained from radiation measured in the prediction interval. A plurality of in-vivo spectra obtained in the prediction interval may be provided. For example, five-hundred (500) in-vivo spectra may be obtained in a prediction interval of 0 to 500 minutes.

In the right side of FIG. 4, the analyte concentration 420 calculated by the calibration model is shown. For example, when 500 in-vivo spectra are input to the calibration model, values of 500 analyte concentrations may be calculated. The values of 500 analyte concentrations may represent analyte concentrations predicted in a prediction interval of 0 to 500 minutes.

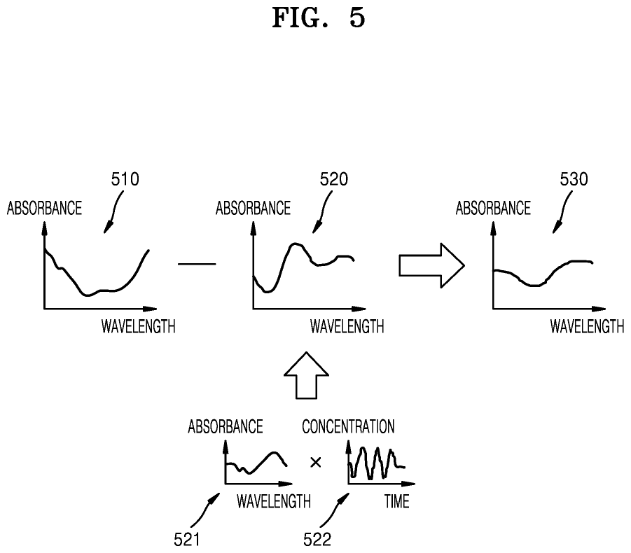

FIG. 5 is a graphical diagram illustrating a method of subtracting a spectrum specific to an analyte from an in-vivo spectrum according to an embodiment.

In the test subject consisting of the analyte and background components, the in-vivo spectrum of the test subject may be interpreted as a combination of a spectrum describing spectral variation by the analyte and a spectrum describing spectral variation by the background components. In the ideal calibration model, it is expected to separately identify the spectrum describing the spectral variation by the analyte and the spectrum describing the spectral variation by the background components.

The spectrum specific to the analyte may be a spectrum describing the spectral variation by the analyte. The spectrum specific to the analyte may be obtained by linear transformation of the net spectrum of the analyte. For example, the spectrum specific to the analyte may be obtained by scaling the net spectrum of the analyte, but is not limited thereto.

In an example calibration model, the spectrum specific to the analyte may be expected to be substantially the same as the spectrum describing the spectral variation by the analyte of the test subject identified by the calibration model. In an example calibration model, the in-vivo spectrum obtained by subtracting the spectrum specific to the analyte therefrom may be expected to be substantially the same as the spectrum describing the spectral variation by the background components of the test subject identified by the calibration model. That is, in an example calibration model, the subtracted in-vivo spectrum and the background spectrum may be expected to be substantially the same.

In one embodiment, the spectrum 520 specific to the analyte may be calculated from the net spectrum 521 of the analyte and the analyte concentration 522 calculated by the calibration model. The spectrum 520 specific to the analyte may be calculated by scaling the net spectrum 521 of the analyte to the analyte concentration 522.

Hₜ(λ)=cₜ*G(λ) [Equation 1]

In Equation 1, t represents an arbitrary time in a prediction section, cₜ represents an analyte concentration corresponding to time t, G(λ) represents a net spectrum of the analyte, and Hₜ(λ) represents a spectrum specific to an analyte calculated by scaling a net spectrum of the analyte to an analyte concentration corresponding to time t.

In one embodiment, a subtracted in-vivo spectrum 530 obtained by subtracting a spectrum 520 specific to the analyte from the in-vivo spectrum 510 may be obtained.

Mₜ(λ)−Hₜ(λ)=Oₜ(λ) [Equation 2]

In Equation 2, t represents an arbitrary time in the prediction interval, Mₜ(λ) represents an in-vivo spectrum corresponding to time t, Hₜ(λ) represents a spectrum specific to an analyte corresponding to time t, and Oₜ(λ) represents a subtracted in-vivo spectrum corresponding to time t.

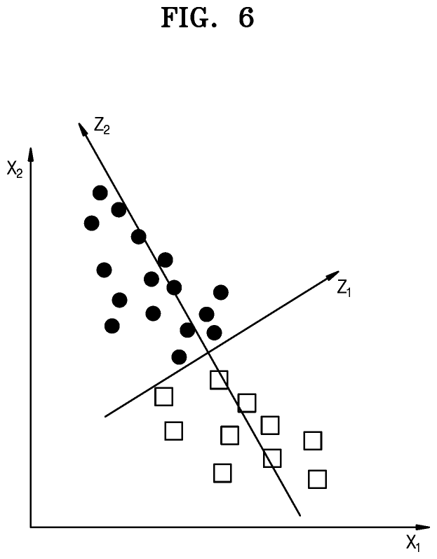

FIG. 6 is a graphical diagram illustrating a feature vector according to an embodiment.

Circles and squares illustrated in FIG. 6 represent arbitrary data M. Coordinate values for the coordinate axes of x₁ and x₂ of M may be features, and feature vectors may be obtained from a set of coordinate values for the coordinate axes of x₁ and x₂. Otherwise, coordinate values for the coordinate axes of z₁ and z₂ of M may be features, and feature vectors may be obtained from a set of coordinate values for the coordinate axes of z₁ and z₂.

In some cases, it may be advantageous for the analysis/use of data M to use feature vectors obtained from coordinate values for the coordinate axes of z₁ and z₂ of data M. Since the z₂ axis has the largest variance when projecting data M among the x₁, x₂, z₁, and z₂ axes, it may be advantageous in terms of distinguishing data M to use coordinate values for the coordinate axes of z₁ and z₂ rather than the coordinate values for the coordinate axes of x₁ and x₂ as feature vectors. In some cases, using a feature vector for the z axis of data M may be advantageous for analysis/use of data M. Circles and squares may be distinguished only by the coordinate values of data M for the z₂ axis. Accordingly, when the feature vector for the z₂ axis is used, the circles and the squares may be most effectively distinguished with the lowest dimension (dimension reduction).

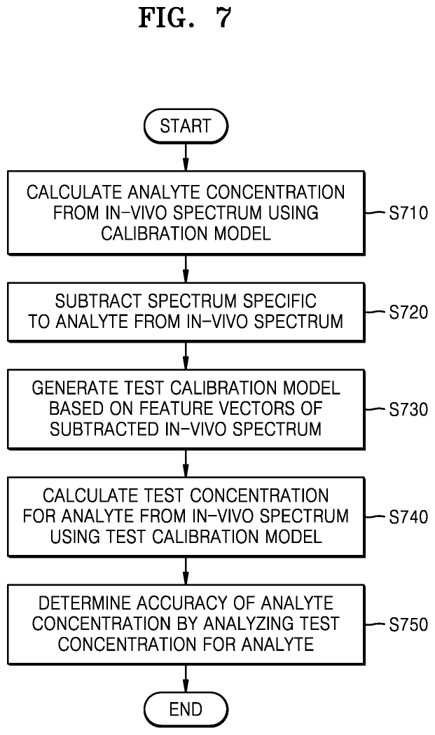

FIG. 7 is a flowchart illustrating a method for determining the accuracy of an analyte concentration model according to an embodiment.

In operation S710, the processor 110 may calculate the analyte concentration from the in-vivo spectrum using the calibration model. The processor 110 may calculate the analyte concentration by executing instructions stored in the memory 120. The in-vivo spectrum may be obtained by the electronic device 100 and provided to the processor 110 or may be provided to the processor 110 from an external device. The analyte concentration may be the concentration of the analyte that is inferred to have been included in the test subject in the prediction interval.

The in-vivo spectrum may be a spectrum obtained by measuring electromagnetic radiation changed by the test subject in the prediction interval. The test subject may be composed of a target analyte and the remaining background components. A test subject may be a living body. The analyte may be a biomolecule of the test subject. The analyte may be at least one of glucose, urea, lactic acid, triglyceride, protein, cholesterol, or ethanol, but is not limited thereto. The in-vivo spectrum may be a combination of the spectrum indicative of a change in radiation by an analyte of a test subject and a spectrum indicative of a change in radiation by background components of the test subject.

The in-vivo spectrum may be an in-vivo spectrum of a test subject obtained based on a spectroscopy. As the spectroscopy, absorption spectroscopy or scattering spectroscopy may be used, but the embodiments are not limited thereto. The in-vivo spectrum may be an in-vivo spectrum of the test subject obtained based on radiation of any wavelength. The radiation may be NIR, SWIR, MWIR, or LWIR, but is not limited thereto.

The calibration model may be a calibration model generated to calculate an analyte concentration from an in-vivo spectrum. The calibration model may be a calibration model generated for a learning interval. The calibration model may be a PLS calibration model, a NAS calibration model, or a calibration model based on deep learning, but is not limited thereto.

In operation S720, the processor 110 may subtract a spectrum specific to the analyte from the in-vivo spectrum. The processor 110 may obtain a spectrum specific to the analyte from the net spectrum of the analyte. The processor 110 may obtain a spectrum specific to the analyte by scaling a net spectrum of the analyte to the analyte concentration calculated in operation S710. In this case, the net spectrum of the analyte used may have units of absorption unit (AU)/concentration. Alternatively, the processor 110 may obtain a spectrum specific to the analyte by scaling a net spectrum of the analyte to an analyte concentration calculated in operation S710 and an optical path length. In this case, the net spectrum of the analyte used may have units of AU/(length*concentration).

The size of the space of the in-vivo spectrum and the size of the space of the spectrum specific to the analyte may be the same. Since the sizes of the spaces are the same, the spectrum specific to the analyte can be subtracted from the in-vivo spectrum. Since the number of spectra in-vivo and the number of analyte concentrations are the same, the number of spectra specific to the analyte obtained by scaling the net spectrum of the analyte to each analyte concentration may be the same as the number of spectra in-vivo. In addition, by using a net spectrum of the analyte for the same wavelength range as the wavelength range of the in-vivo spectrum, the wavelength range of the spectra specific to the analyte may be the same as the wavelength range of the in-vivo spectrum. For example, assuming in-vivo spectra of N wavelength ranges of A to B, N analyte concentrations are calculated by the calibration model. When the net spectrum of an analyte in the wavelength range of A to B is multiplied by the N analyte concentrations, N spectra specific to the analyte in the wavelength range of A to B may be obtained.

In operation S730, the processor 110 may generate a test calibration model based on feature vectors of the subtracted in-vivo spectrum.

The processor 110 may calculate feature vectors of the subtracted in-vivo spectrum. The processor 110 may calculate feature vectors by performing feature extraction from the subtracted in-vivo spectrum. Various techniques may be used for feature extraction. The feature extraction may use, but is not limited to, Principal Component Analysis (PCA), Linear Discriminant Analysis (LDA), Singular Value Decomposition (SVD), Independent Component Analysis (ICA), Eigenvalue Decomposition (EVD), Non-Negative Matrix Factorization (NMF), Locally Linear Embedding (LLE), Locality Preserving Projection (LPP), Unsupervised Discriminant Projection (UDP), or Factor Analysis (FA). For example, when the PCA is used for feature extraction, principal components may be calculated as feature vectors.

As a test calibration model is generated using feature vectors of the subtracted in-vivo spectrum, the test calibration model may be generated with low-dimensional data with reduced noise. In addition, by generating a test calibration model using feature vectors of the subtracted in-vivo spectrum, the test calibration model may be generated using the principal components of the subtracted in-vivo spectrum.

The test calibration model may be generated to calculate the test concentration of the analyte from the in-vivo spectrum. The test calibration model may be a NAS calibration model. The NAS calibration model may be generated by calculating a background spectrum and a NAS from the in-vivo spectrum and the net spectrum of the analyte, and in operation S730, the processor 110 may generate a test calibration model using the feature vectors of the subtracted in-vivo spectrum as a background spectrum. That is, the test calibration model may be generated to calculate the NAS using feature vectors of the subtracted in-vivo spectrum and the net spectrum of the analyte.

In operation S740, the processor 110 may calculate the test concentration of the analyte from the in-vivo spectrum using the test calibration model. If the accuracy of the analyte concentration is high due to the excellent performance of the calibration model, the spectrum specific to the analyte generated based on the analyte concentration is expected to be substantially the same as the spectrum describing the spectral variation by the analyte of the test subject. In other words, the in-vivo spectrum from which the spectrum specific to the analyte is subtracted is expected to be substantially the same as the spectrum describing the spectral variation by the background components of the test subject. In other words, the test concentration calculated by the test calibration model generated using the subtracted in-vivo spectrum is expected to be substantially the same as the analyte concentration calculated by the calibration model.

The in-vivo spectrum for the prediction interval may be used to calculate the test concentration. When the in-vivo spectrum for the prediction interval is used, a test concentration for the prediction interval may be calculated. For example, when the prediction interval is 0 to 500 minutes, a test concentration for 0 to 500 minutes may be calculated. Alternatively, an in-vivo spectrum for a sub-interval of the prediction interval may be used to calculate the test concentration. For example, when the prediction interval is 0 to 500 minutes and the sub-interval is 0 to 10 minutes, a test concentration for 0 to 10 minutes may be calculated.

In operation S750, the processor 110 may determine the accuracy of the analyte concentration by analyzing the test concentration of the analyte. The processor 110 may determine the accuracy of the analyte concentration by comparing the test concentration with a reference value. The processor 110 may determine the accuracy of the analyte concentration by comparing the standard deviation of the test concentration with a reference value. The processor 110 may determine the accuracy of the analyte concentration by comparing the test concentration for the prediction interval with a reference value. The processor 110 may determine the accuracy of the analyte concentration by comparing the test concentration for the sub-interval with a reference value. The processor 110 may determine the accuracy of the analyte concentration by comparing the standard deviation of the test concentration for the sub-interval with a reference value. For example, the processor 110 may determine that the accuracy of the analyte concentration is high if the standard deviation of the test concentration is less than the reference value, and that the accuracy of the analyte concentration is low if the standard deviation of the test concentration is not less than the reference value. For example, the reference value for comparison with the standard deviation of the test concentration may be, but is not limited to, a value included in the range of about 1 mg/dl to about 1000 mg/dl. For example, the processor 110 may determine that the accuracy of the analyte concentration is high if the standard deviation of the test concentration for the subinterval 0-10 minutes is less than 50 mg/dl, and that the accuracy of the analyte concentration is low if it is not less than 50 mg/dl.

The processor 110 may determine the accuracy of the analyte concentration by comparing the test concentration with the analyte concentration. When the performance of the calibration model is excellent, it is expected that the test concentration calculated by the test correction model and the concentration calculated by the calibration model are substantially the same. The processor 110 may determine the accuracy of the analyte concentration by comparing the difference between the test concentration and the analyte concentration with a reference value. The processor 110 may determine the accuracy of the analyte concentration by comparing the difference between the test concentration and the analyte concentration for the prediction interval with a reference value. The processor 110 may determine the accuracy of the analyte concentration by comparing the difference between the test concentration and the analyte concentration for the sub-interval with a reference value. For example, the reference value may be determined from the difference between the maximum value and the minimum value of the analyte concentration. For example, the reference value may be a value obtained by scaling the difference between the maximum value and the minimum value of the analyte concentration to 0.1.

According to the method for determining the accuracy of the analyte concentration described with reference to FIG. 7, an invasive method is not required to determine the accuracy of the analyte concentration calculated for the prediction interval. In other words, using the method for determining the accuracy of the analyte concentration according to the present disclosure, it is possible to determine the accuracy of the analyte concentration calculated for the predicted interval in a non-invasive manner.

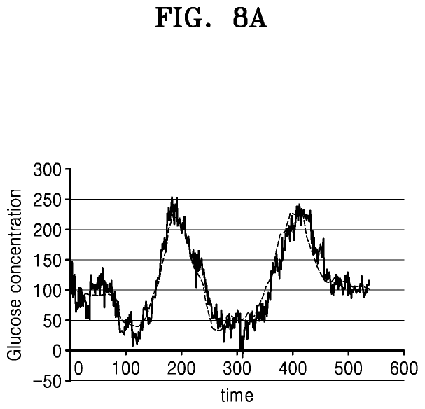

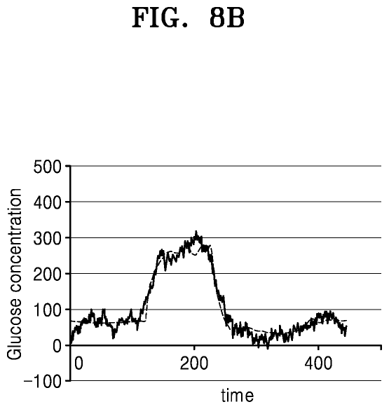

FIGS. 8A and 8B illustrate graphs of concentrations and test concentrations of glucose calculated by experiments according to an embodiment.

FIG. 8A shows the results of a first experiment, and FIG. 8B shows the results of a second experiment. The analytes of the first and second experiments are glucose. The first experiment and the second experiment were performed on different prediction intervals. In the graphs, the dotted lines represent the concentrations of glucose calculated by the calibration model, and the solid lines represent the test concentrations of glucose calculated by the test calibration model.

Referring to FIGS. 8A and 8B, a difference between a glucose concentration calculated by a calibration model for a prediction interval and a test concentration of glucose calculated by a test calibration model is less than a reference value. In addition, the standard deviation of the test concentration of glucose is less than the reference value. According to the method shown in FIG. 7, the processor 110 determines that the accuracy of the glucose concentration calculated by the calibration model is high from the experimental result of FIGS. 8A and 8B. Referring to the graphs in FIGS. 8A and 8B, the difference between the glucose concentration calculated by the calibration model and the test concentration calculated by the test calibration model is not significant in the prediction interval, so the processor 110 may determine the accuracy of the glucose concentration calculated by the calibration model by analyzing the test concentration of glucose in the sub-interval of the prediction interval.

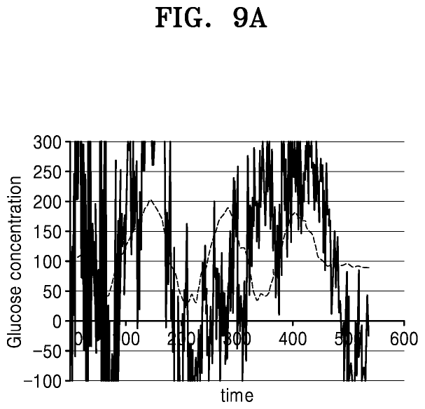

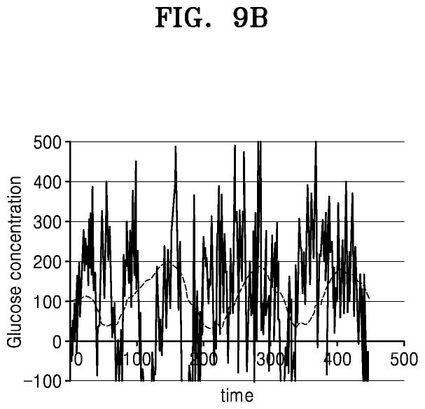

FIGS. 9A and 9B illustrate graphs of concentrations and test concentrations of glucose calculated by experiments according to an embodiment.

FIG. 9A shows the result of a third experiment, and FIG. 9B shows the result of a fourth experiment. The analytes of the third and fourth experiments are glucose. The third and fourth experiments were performed on different prediction intervals. In the graphs, the dotted lines represent the concentrations of glucose calculated by the calibration model, and the solid lines represent the test concentrations of glucose calculated by the test calibration model.

Referring to FIGS. 9A and 9B, a difference between a glucose concentration calculated by a calibration model and a test concentration of glucose calculated by a test calibration model for a prediction interval is greater than a reference value. In addition, the standard deviation of the test concentration of glucose is greater than the reference value. According to the method shown in FIG. 7, the processor 110 determines that the accuracy of the glucose concentration calculated by the calibration model is low from the experimental results of FIGS. 9A and 9B. Referring to FIGS. 9A and 9B, the difference between the glucose concentration calculated by the calibration model and the test concentration calculated by the test calibration model is significant in the prediction interval.

Meanwhile, the method described above may be recorded in a computer-readable non-transitory recording medium in which one or more programs including instructions for executing the method are recorded. Examples of computer-readable recording media include magnetic media, such as hard disks, floppy disks, and magnetic tapes, optical media, such as CD-ROMs and DVDs, magneto-optical media, such as floptical disks, and hardware devices, such as ROMs, RAMs, and flash memories, which are particularly configured to store and perform program instructions. Examples of program instructions include machine language codes, such as those produced by compilers, as well as advanced language codes that can be executed by computers using interpreters, etc.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims and their equivalents.

What is claimed is:

1. An electronic device for determining accuracy of an analyte concentration non-invasively, the electronic device comprising:
a memory configured to store one or more instructions; and
one or more processors, wherein the one or more processors are configured to execute the one or more instructions to:
calculate an analyte concentration of an analyte from an in-vivo spectrum obtained non-invasively by using a calibration model;
subtract a spectrum specific to the analyte from the in-vivo spectrum;
generate a test calibration model based on feature vectors of the subtracted in-vivo spectrum obtained non-invasively;
calculate a test concentration for the analyte from the in-vivo spectrum by using the test calibration model; and
determine the accuracy of the analyte concentration calculated using the calibration model by analyzing the test concentration for the analyte using the test calibration model.

2. The electronic device of claim 1, wherein the one or more processors are configured to obtain the spectrum specific to the analyte by scaling a net spectrum of the analyte to the analyte concentration.

3. The electronic device of claim 1, wherein the one or more processors are configured to obtain the spectrum specific to the analyte by scaling a net spectrum of the analyte to the analyte concentration and an optical path length.

4. The electronic device of claim 1, wherein the one or more processors are configured to generate the test calibration model by calculating a net analyte signal (NAS) from feature vectors of the subtracted in-vivo spectrum.

5. The electronic device of claim 4, wherein the one or more processors are configured to obtain the feature vectors by performing feature extraction on the subtracted in-vivo spectrum.

6. The electronic device of claim 5, wherein the feature vectors of the subtracted in-vivo spectrum comprise main components obtained by performing principal component analysis (PCA) on the subtracted in-vivo spectrum.

7. The electronic device of claim 1, wherein the one or more processors are configured to determine the accuracy of the analyte concentration by comparing the test concentration for the analyte with a reference value.

8. The electronic device of claim 1, wherein the one or more processors are configured to determine the accuracy of the analyte concentration by comparing, with a reference value, a standard deviation of the test concentration for the analyte.

9. The electronic device of claim 1, wherein the one or more processors are configured to determine the accuracy of the analyte concentration by comparing, with a reference value, a standard deviation of the test concentration calculated for a predetermined interval.

10. The electronic device of claim 1, wherein the one or more processors are configured to determine that the accuracy of the analyte concentration is high when a standard deviation of the test concentration calculated for a 10-minute interval is less than 50 mg/dl.

11. The electronic device of claim 1, wherein the one or more processors are configured to determine the accuracy of the analyte concentration by comparing the test concentration for the analyte with the analyte concentration.

12. The electronic device of claim 1, wherein the calibration model is at least one of a partial least squares (PLS) calibration model, a net analyte signal (NAS) calibration model, and a calibration model based on deep learning.

13. The electronic device of claim 1, wherein the test calibration model is a NAS calibration model.

14. The electronic device of claim 1, wherein the analyte is at least one of glucose, urea, lactic acid, triglyceride, protein, cholesterol, and ethanol.

15. The electronic device of claim 1, wherein the in-vivo spectrum is an in-vivo spectrum of a test subject generated based on absorption spectroscopy.

16. The electronic device of claim 1, wherein the in-vivo spectrum is an in-vivo spectrum of a test subject for near-infrared rays.

17. The electronic device of claim 1, wherein the one or more processors are configured to determine, in a non-invasive manner, the accuracy of the analyte concentration calculated using the calibration model.

18. A method of determining accuracy of an analyte concentration non-invasively, the method comprising:
calculating the analyte concentration from an in-vivo spectrum obtained non-invasively by using a calibration model;
subtracting a spectrum specific to an analyte from the in-vivo spectrum;
generating a test calibration model based on feature vectors of the subtracted in-vivo spectrum obtained non-invasively;
calculating a test concentration for the analyte from the in-vivo spectrum by using the test calibration model; and
analyzing the test concentration for the analyte using the test calibration model to determine accuracy of the analyte concentration non-invasively.

19. The method of claim 18, wherein the determining of the accuracy of the analyte concentration comprises comparing the test concentration of the analyte with a reference value to determine the accuracy of the analyte concentration.

20. A computer-readable recording medium having recorded thereon a program for executing the method of claim 19 on a computer.
